(19) United States
(12) Patent Application Publication
(10) Pub. No.: US 2025/0009236 A1
(43) Pub. Date: Jan. 9, 2025

(54) INTRACARDIAC PRESSURE ESTIMATION DEVICE, INTRACARDIAC PRESSURE ESTIMATION SYSTEM, INTRACARDIAC PRESSURE ESTIMATION METHOD, AND PROGRAM

(71) Applicant: TERUMO KABUSHIKI KAISHA, Tokyo (JP)

(72) Inventors: Takayuki UCHIDA, Hiratsuka-shi (JP); Tomonori HATTA, Hadano-shi (JP); Ryo ICHIKAWA, Atsugi-shi (JP)

(73) Assignee: TERUMO KABUSHIKI KAISHA, Tokyo (JP)

(21) Appl. No.: 18/897,378

(22) Filed: Sep. 26, 2024

Related U.S. Application Data
(63) Continuation of application No. PCT/JP2023/007700, filed on Mar. 1, 2023.

(30) Foreign Application Priority Data
Mar. 28, 2022 (JP) 2022-052135

(51) Int. Cl. A61B 5/0205; A61B 5/00; A61B 5/021; A61B 5/346; A61B 7/04

(52) U.S. Cl. CPC A61B 5/0205; A61B 5/7275; A61B 5/7278; A61B 5/021; A61B 5/346; A61B 7/04; A61B 2505/07

(57) ABSTRACT

An intracardiac pressure estimation device includes an acquisition unit, an input unit, and a control unit. The acquisition unit obtains cardiac function-related information related to a cardiac function. The input unit receives an input of load information indicating a load applied to a body of a person to be measured when the cardiac function-related information is measured. The control unit estimates an intracardiac pressure, which is a pressure applied to the inside of a heart or to a blood vessel near the heart, on the basis of the cardiac function-related information, obtains variation factor information, which is information indicating a variation factor that may affect the intracardiac pressure and includes the load information, and calculates an adjusted value of the intracardiac pressure according to the variation factor information.

### CROSS-REFERENCES TO RELATED APPLICATIONS

This application is a continuation of International Application No. PCT/JP2023/007700 filed on Mar. 1, 2023, which claims priority to Japanese Application No. 2022-052135 filed on Mar. 28, 2022, the entire content of both of which is incorporated herein by reference.

### TECHNOLOGICAL FIELD

The present disclosure relates to an intracardiac pressure estimation device, an intracardiac pressure estimation system, an intracardiac pressure estimation method, and a program.

### BACKGROUND DISCUSSION

It is known that, in order to reduce a rehospitalization rate of a heart failure patient, it is effective to estimate and monitor an intracardiac pressure, such as a left ventricular end-diastolic pressure (LVEDP) and a pulmonary arterial pressure (PAP), by non-invasive measurement at home. Intracardiac pressures such as atrial pressure, ventricular pressure, and pulmonary arterial pressure, blood pressure, cardiac output, and the like are common indices of hemodynamics. In addition, hemodynamics can fluctuate due to an influence of a load caused, for example, by diet, exercise, and the like. U.S. Pat. No. 7,946,995 discloses that it is preferable to consider an activity cycle of a patient at a time of analyzing hemodynamics.

U.S. Pat. No. 7,946,995 discloses that a left atrial pressure (LAP) increases during daytime activity due to an increase in a load on a cardio-respiratory system and decreases during nighttime inactivity, such as sleep, as the load decreases. In addition, according to the invention disclosed in U.S. Pat. No. 7,946,995, it is determined that there is an abnormality in a heart condition when a difference between the daytime LAP and the nighttime LAP is smaller than a predetermined value.

Since the load on the body of the patient affects the intracardiac pressure, it is ideally desirable to measure the intracardiac pressure of the heart failure patient under a constant condition at all times. However, in the case of estimating the intracardiac pressure by the non-invasive measurement at home or the like, it is difficult to perform measurement under the same conditions in all measuring actions. When the measurement conditions vary, it becomes difficult to correctly evaluate the measured intracardiac pressure. In view of the above, it is preferable that a risk of heart failure can be determined in consideration of variation factor information, which includes the load applied to the body of the patient and affects the intracardiac pressure.

### SUMMARY

An intracardiac pressure estimation device, an intracardiac pressure estimation system, an intracardiac pressure estimation method, and a program are disclosed that enables evaluation of an intracardiac pressure in consideration of an influence of variation factors including a load applied to a body of a person to be measured.

An intracardiac pressure estimation device according to an aspect of the present disclosure includes an acquisition unit that obtains cardiac function-related information related to a cardiac function, an input unit that receives an input of load information indicating a load applied to a body of a person to be measured when the cardiac function-related information is measured, and a control unit that estimates an intracardiac pressure that is a pressure applied to an inside of a heart or to a blood vessel near the heart on the basis of the cardiac function-related information, obtains variation factor information that is information indicating a variation factor that may affect the intracardiac pressure and includes the load information, and calculates an adjusted value of the intracardiac pressure according to the variation factor information.

As an embodiment, the acquisition unit obtains the cardiac function-related information measured by a non-invasive means.

As an embodiment, the cardiac function-related information includes at least one of an electrocardiogram, a pulse wave, and a heart sound.

As an embodiment, the intracardiac pressure includes at least one of a left ventricular end-diastolic pressure, a pulmonary arterial pressure, and a pulmonary wedge pressure.

As an embodiment, the load information includes at least one of biological information of the person to be measured, environmental information, and presence or absence of a daily life activity of the person to be measured and a time elapsed after the activity is performed.

As an embodiment, the control unit calculates the adjusted value by using an adjustment amount determined on the basis of at least one of a type and magnitude of the load included in the load information for adjustment of an estimated value of the intracardiac pressure estimated on the basis of the cardiac function-related information.

As an embodiment, the variation factor information includes time variation information indicating a variation in the intracardiac pressure based on time, and the time variation information includes variation information of at least one of an intra-day variation, a daily variation, a weekly variation, a monthly variation, a seasonal variation, and a yearly variation.

As an embodiment, the time variation information is constructed by the intracardiac pressure of the person to be measured accumulated together with time information.

As an embodiment, the time variation information includes the variation information of the intra-day variation, and the control unit determines that a disease risk is high when the intra-day variation is smaller than a predetermined threshold.

As an embodiment, the control unit determines a disease risk on the basis of the adjusted value of the intracardiac pressure.

As an embodiment, the control unit considers a weight of the person to be measured input to the input unit when the disease risk is determined.

As an embodiment, the intracardiac pressure estimation device is used by the person to be measured who has been discharged from a hospital after heart failure treatment, and the control unit considers a period of time elapsed after the discharge of the person to be measured input to the input unit when the disease risk is determined.

As an embodiment, the control unit determines a schedule for measuring the cardiac function-related information on the basis of the determined disease risk.

An intracardiac pressure estimation system according to an aspect of the present disclosure includes a measurement device that measures cardiac function-related information related to a cardiac function, and an intracardiac pressure estimation device including an acquisition unit that obtains the cardiac function-related information, an input unit that receives an input of load information indicating a load applied to a body of a person to be measured when the cardiac function-related information is measured, and a control unit that estimates an intracardiac pressure that is a pressure applied to an inside of a heart or to a blood vessel near the heart on the basis of the cardiac function-related information, obtains variation factor information that is information indicating a variation factor that may affect the intracardiac pressure and includes the load information, and calculates an adjusted value of the intracardiac pressure according to the variation factor information.

An intracardiac pressure estimation method according to an aspect of the present disclosure is an intracardiac pressure estimation method to be executed by a control unit of a computer, the method including obtaining cardiac function-related information related to a cardiac function, guiding an input of load information to an input unit, the load information indicating a load applied to a body of a person to be measured when the cardiac function-related information is measured, receiving the input of the load information from the input unit, estimating an intracardiac pressure that is a pressure applied to an inside of a heart or to a blood vessel near the heart on the basis of the cardiac function-related information, and calculating an adjusted value of the intracardiac pressure according to variation factor information that is information indicating a variation factor that may affect the intracardiac pressure and includes the load information.

A non-transitory computer-readable medium storing a program according to an aspect of the present disclosure causes a processor to execute a process including obtaining cardiac function-related information related to a cardiac function, guiding an input of load information to an input unit, the load information indicating a load applied to a body of a person to be measured when the cardiac function-related information is measured, receiving the input of the load information from the input unit, estimating an intracardiac pressure that is a pressure applied to an inside of a heart or to a blood vessel near the heart on the basis of the cardiac function-related information, and calculating an adjusted value of the intracardiac pressure according to variation factor information that is information indicating a variation factor that may affect the intracardiac pressure and includes the load information.

According to the present disclosure, the adjusted value of the intracardiac pressure is calculated according to the variation factor information including the load information, whereby the intracardiac pressure may be evaluated in consideration of the influence of the load applied to the body of the person to be measured.

### BRIEF DESCRIPTION OF THE DRAWINGS

FIG. 1 is a block diagram illustrating a schematic configuration of an intracardiac pressure estimation system according to an embodiment.

FIG. 2 is a diagram illustrating an example of components included in a storage unit in FIG. 1.

FIG. 3 is a functional block diagram illustrating a process to be performed by a control unit in FIG. 1.

FIG. 4 is a diagram illustrating an example of variation in intracardiac pressure.

FIG. 5 is a flowchart illustrating an example of the process to be performed by the control unit in FIG. 1.

### DETAILED DESCRIPTION

Set forth below with reference to the accompanying drawings is a detailed description of embodiments of an intracardiac pressure estimation device, an intracardiac pressure estimation system, an intracardiac pressure estimation method, and a program.

### Intracardiac Pressure Estimation System

An intracardiac pressure estimation system 1 of the present disclosure is a system that estimates an intracardiac pressure of a person to be measured using one or more measurement devices and adjusts the estimated intracardiac pressure to an adjusted value under a predetermined condition in consideration of a difference in measurement conditions. The intracardiac pressure estimation system 1 can be used at home by a user who does not necessarily have medical expertise, such as the person to be measured him/herself discharged from a hospital after heart failure treatment, a close relative of the person to be measured, or the like. Note that, in the present disclosure, the “adjusted value” indicates a value converted into a value that would have been measured if the value had been measured under the predetermined conditions.

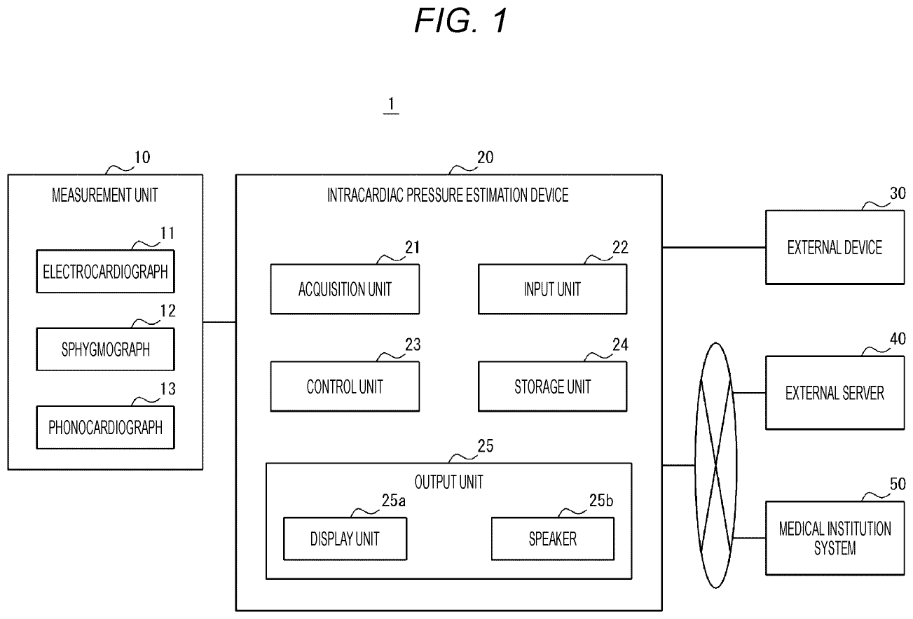

As illustrated in FIG. 1, the intracardiac pressure estimation system 1 of the present disclosure includes a measurement unit 10 and an intracardiac pressure estimation device 20. The measurement unit 10 and the intracardiac pressure estimation device 20 may be individual devices mounted on different pieces of hardware. In that case, the measurement unit 10 and the intracardiac pressure estimation device 20 are connected by wired and/or wireless communication means. In another embodiment, all or some of functions of the measurement unit 10 and the intracardiac pressure estimation device 20 may be mounted on the same hardware.

### Configuration of Measurement Unit

The measurement unit 10 is one or more measurement devices that measure cardiac function-related information related to a cardiac function of the person to be measured. The cardiac function-related information includes information related to at least one of heartbeat and blood pressure. The measurement unit 10 may include a non-invasive measurement means. The non-invasive measurement means can include, for example, an electrocardiogra sphygmograph 12, and a phonocardiograph 13. The measurement unit 10 may include all of the electrocardiogramaphygmograph 12, and the phonocardiograph 13, or may include only some of them. The measurement unit 10 may further include another measurement device, such as a sphygmomanometer. The measurement unit 10 may also include an invasive measurement device, such as a catheter for invasive arterial pressure measurement. The measurement unit 10 may transmit a measurement result to the intracardiac pressure estimation device 20 as electrical signals.

The electrocardiogramcludes an electrode to be attached to a part such as a wrist, ankle, chest, or the like to obtain an electrocardiogram of a user. The electrode may detect minute electricity generated in the heart. In one embodiment, the electrical signals detected by the electrode of the electrocardiograph 11 are processed by a main body part of the electrocardiogra 11, which is separate from the intracardiac pressure estimation device 20, and the measurement result is transmitted to the intracardiac pressure estimation device 20. In another embodiment, the electrical signals detected by the electrode of the electrocardiograph be directly transmitted to the intracardiac pressure estimation device 20, and may be processed in the intracardiac pressure estimation device 20.

The sphygmograph 12 compresses a blood vessel by sending air into an arm cuff wound around an upper arm or the like of the user, and measures a pulse wave transmitted to the blood vessel by the heartbeat. In one embodiment, the arm cuff is controlled by a main body part of the sphygmograph 12, which is separate from the intracardiac pressure estimation device 20, and performs measurement. In another embodiment, the arm cuff of the sphygmograph 12 may be directly connected to the intracardiac pressure estimation device 20, and may be controlled by the intracardiac pressure estimation device 20 to perform measurement. The sphygmograph 12 may measure the pulse wave with a tonometer, a pressure sensor, or the like instead of the arm cuff.

The phonocardiograph 13 measures a heartbeat sound of the heart of the user with a microphone. Examples of the microphone can include a condenser microphone that detects a change in capacitance between a diaphragm (vibration plate) and a backplate (electrode), a piezoelectric microphone using a piezoelectric element, and an electrodynamic microphone in which a permanent magnet and a coil are combined. In one embodiment, the microphone of the phonocardiograph 13 is controlled by a main body part of the phonocardiograph 13, which is separate from the intracardiac pressure estimation device 20, and performs measurement. In another embodiment, the microphone of the phonocardiograph 13 may be directly connected to the intracardiac pressure estimation device 20, and may be controlled by the intracardiac pressure estimation device 20 to perform measurement. The phonocardiograph 13 may measure an acceleration level or a body-conducted sound, or may use a piezoelectric element instead of the microphone.

### Configuration of Intracardiac Pressure Estimation Device

The intracardiac pressure estimation device 20 includes an acquisition unit 21, an input unit 22, a control unit 23, a storage unit 24, and an output unit 25. The intracardiac pressure estimation device 20 is a computer equipped with a program for estimating intracardiac pressure. The intracardiac pressure estimation device 20 may be, for example, any of a dedicated computer device, a general-purpose computer such as a personal computer (PC), or a server device such as a PC server, or a workstation.

The “intracardiac pressure” in the present application indicates pressure inside the heart or pressure applied to a blood vessel near the heart. The intracardiac pressure can include, for example, a left ventricular end-diastolic pressure (LVEDP), a pulmonary arterial pressure (PAP), and a pulmonary wedge pressure (PWP), and further includes a left/right atrial pressure, a left/right ventricular pressure, an arterial pressure, and the like, but is not limited to the examples as set forth above. The pulmonary wedge pressure is also called a pulmonary arterial wedge pressure (PAWP), a pulmonary capillary wedge pressure (PCWP), or a pulmonary artery occlusion pressure (PAOP). The intracardiac pressure may be used as an indicator of a sign of exacerbation of heart failure.

The acquisition unit 21 obtains, from the measurement unit 10, the cardiac function-related information measured by the measurement unit 10. The acquisition unit 21 may obtain each piece of the cardiac function-related information in the measurement unit 10 as electrical signals. The acquisition unit 21 may receive detection signals before signal processing from the measurement unit 10, or may receive a measurement value subjected to the signal processing by the measurement unit 10. In the former case, the signal processing may be performed in the intracardiac pressure estimation device 20 to calculate the measurement value. The acquisition unit 21 may include an input terminal that receives an input from the measurement unit 10, and/or a communication interface with the measurement unit 10.

The input unit 22 is an input interface that receives inputs of various types of information to the intracardiac pressure estimation device 20 from the outside of the intracardiac pressure estimation device 20.

The input unit 22 can include one or more input interfaces that detect an input made by the user and obtain input information based on the user operation. For example, the input unit 22 may include a physical key, a capacitance key, a touch screen disposed integrally with a display unit 25a of the output unit 25 to be described later, a microphone that receives an audio input, or the like.

The input unit 22 may include an input interface configured to receive input signals from an external device 30. The input unit 22 may include a wireless communication device and a connector for inputting data to the intracardiac pressure estimation device 20. The external device 30 can include, for example, devices such as a body motion sensor that measures load information indicating a load applied to the body of the person to be measured, an activity meter, a blood sugar meter, a thermometer, and the like. The external device 30 may further include a device that measures information regarding the person to be measured, such as a scale.

The input unit 22 may include a communication interface for receiving input from a remotely located information device and/or system via a communication line. The remotely located information device and system can include, for example, an external server 40 and/or a medical institution system 50. The input unit 22 may correspond to the wired and/or wireless communication means.

The external server 40 is a server device that collects and/or manages information such as a measurement value of the cardiac function-related information and an estimated value and an adjusted value of the intracardiac pressure from the intracardiac pressure estimation device 20, and is a device that provides information to the intracardiac pressure estimation device 20. The information provided by the external server 40 can include, for example, an estimated value of the previous intracardiac pressure of the person to be measured, intra-day variation data of the intracardiac pressure of a general person, and the like.

The medical institution system 50 is a system of a medical institution at which the person to be measured receives medical treatment as a patient. The intracardiac pressure estimation device 20 may be configured to receive information related to the medical treatment of the patient from the medical institution system 50. The information related to the medical treatment of the patient may include electronic medical record information, discharge date information indicating a date of discharge from the hospital after the heart failure treatment, and the like. The medical institution system 50 may receive the measurement value of the cardiac function-related information of the person to be measured and/or the estimated value of the intracardiac pressure from the intracardiac pressure estimation device 20. Those pieces of information may be used as a material to be considered by a doctor to determine a condition of the patient, who is the person to be measured, and to change a prescription or the like in the medical institution.

The control unit 23 can include at least one processor, at least one dedicated circuit, or a combination of at least one processor and at least one dedicated circuit. The processor can be a general-purpose processor such as a central processing unit (CPU) or a graphics processing unit (GPU), or a dedicated processor specialized for specific processing. The dedicated circuit can be, for example, a field-programmable gate array (FPGA) or an application specific integrated circuit (ASIC).

The control unit 23 executes processing related to operation of the intracardiac pressure estimation device 20 while controlling each unit of the intracardiac pressure estimation device 20. The processing to be executed by the control unit 23 will be described later.

The storage unit 24 can be, for example, a semiconductor memory, a magnetic memory, an optical memory, or the like, but is not limited to a semiconductor magnetic memory, an optical memory, or the like. The storage unit 24 may function as, for example, a main storage device, an auxiliary storage device, or a cache memory. The storage unit 24 stores any information to be used to operate the intracardiac pressure estimation device 20. For example, the storage unit 24 may sequentially store a system program, an application program, information obtained by the intracardiac pressure estimation device 20, and the like. A part of the storage unit 24 may be installed outside the intracardiac pressure estimation device 20. In that case, the part of the storage unit 24 installed outside may be coupled to the intracardiac pressure estimation device 20 via any interface.

The storage unit 24 may sequentially store information input from the input unit 22 under the control of the control unit 23. The storage unit 24 may sequentially update the stored information with new information.

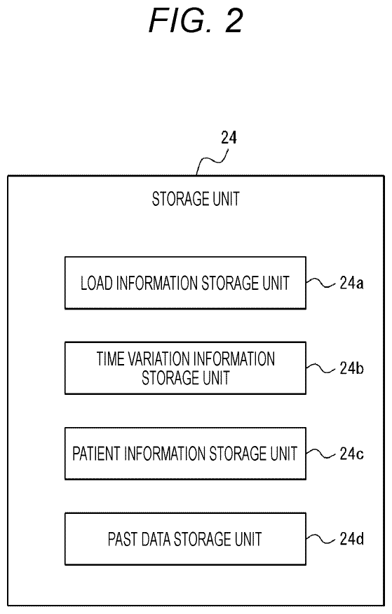

As illustrated in FIG. 2, the storage unit 24 may include a load information storage unit 24a, a time variation information storage unit 24b, a patient information storage unit 24c, and a past data storage unit 24d. Each unit of the storage unit 24 may store information as a database managed by a database management system or as a file describing data.

The load information storage unit 24a stores load information, which is input from the input unit 22 and is information related to a load applied to the body of the person to be measured. The load information includes at least one of biological information of the person to be measured, environmental information, and presence or absence of a daily life activity of the person to be measured and a time elapsed after the activity is performed. The biological information of the person to be measured includes a physical activity amount, an acceleration level, a blood glucose level, a heart rate, a blood pressure, a body temperature, sleep hours, and an autonomic nervous activity. The daily life activity of the person to be measured includes bathing, exercise, dosing, diet, drinking, smoking, urination, and defecation. The environmental information includes a temperature and a humidity around the person to be measured. The autonomic nervous activity includes various parameters calculated by heart rate variability analysis or the like, and can include, for example, parameters such as a low frequency (LF)/high frequency (HF) ratio that indicates a balance between the sympathetic nervous system and the parasympathetic nervous system, total power that indicates a total amount of the autonomic nervous activities of the sympathetic nervous system and the parasympathetic nervous system, and the like. The storage unit 24 may store some or all of those pieces of load information.

The time variation information storage unit 24b stores time variation information, which is information indicating time variation of the estimated value of the intracardiac pressure of the person to be measured. The time variation information includes each piece of variation information such as an intra-day variation, a daily variation, a weekly variation, a monthly variation, a seasonal variation, and a yearly variation. The storage unit 24 may sequentially store and accumulate the estimated value of the intracardiac pressure estimated by the intracardiac pressure estimation device 20 together with time information. The time information can include, for example, the time at which the cardiac function-related information related to the cardiac function is measured or obtained, the time at which the intracardiac pressure is estimated, or the time at which the adjusted value of the intracardiac pressure is calculated. The time variation information may be constructed by the control unit 23 statistically processing the information regarding the estimated value of the intracardiac pressure accumulated in the storage unit 24. Furthermore, the control unit 23 may obtain time variation of intracardiac pressure of an average person from the external server 40, and may construct the time variation information using the obtained time variation.

The patient information storage unit 24c stores, as patient information, information regarding the person to be measured obtained by the input unit 22 from the external device 30, such as a scale, and the medical institution system 50. The patient information may include a measurement value such as a weight of the person to be measured, electronic medical record information, discharge date information, and the like.

The past data storage unit 24d sequentially stores, as past data, at least one or more of the measurement value of the cardiac function-related information, the estimated value of the intracardiac pressure, and the adjusted value of the intracardiac pressure obtained from the measurement unit 10. The past data is used for data analysis, generation of time variation information, disease risk determination for the person to be measured, and the like performed by the control unit 23.

The output unit 25 includes one or more output interfaces that output information and notify the user. For example, the output unit 25 can include the display unit 25a, which is a display that outputs information as an image, and/or a speaker 25b that outputs information as a sound, etc., but is not limited to a display unit 25a and/or a speaker 25b. The output unit 25 may be configured to output information in various modes. Under the control of the control unit 23, the output unit 25 is enabled to display information for guiding an input of the load information, a measurement schedule, information for making notification regarding measurement time, and the like. The output unit 25 may further include a communication interface for transmitting information to a remote device, such as the external server 40 and the medical institution system 50.

### Operation of Control Unit

The functions of the intracardiac pressure estimation device 20 are implemented by a processor, which serves as the control unit 23, executing the program according to the present embodiment. That is, the functions of the intracardiac pressure estimation device 20 are implemented by software. The program causes the processor of the computer to execute the operation of the intracardiac pressure estimation device 20, thereby causing the computer to function as the intracardiac pressure estimation device 20.

The program may be stored in a non-transitory computer-readable medium. Examples of the non-transitory computer-readable medium include a flash memory, a magnetic recording device, an optical disk, a magneto-optical recording medium, and a read-only memory (ROM). The program can be distributed by, for example, selling, transferring, or lending a portable medium such as a secure digital (SD) card, a digital versatile disc (DVD), or a compact disc read only memory (CD-ROM) storing the program. The program may be distributed by being stored in storage of a server in advance and transferred from the server to another computer. The program may be provided as a program product.

Some or all of the functions of the intracardiac pressure estimation device 20 may be implemented by a programmable circuit or a dedicated circuit serving as the control unit 23. That is, some or all of the functions of the intracardiac pressure estimation device 20 may be implemented by hardware.

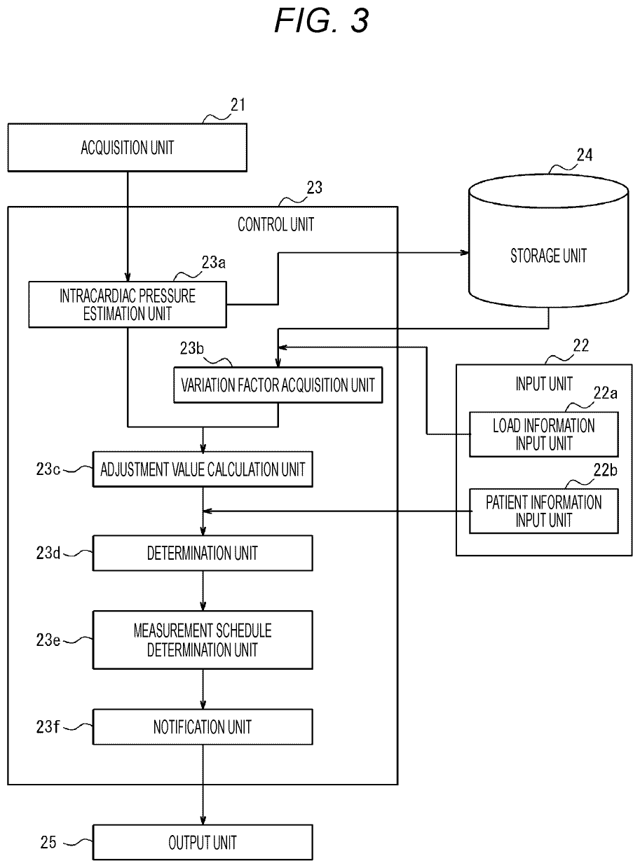

A process to be executed by the control unit 23 will be described with reference to FIG. 3. The control unit 23 includes individual components of an intracardiac pressure estimation unit 23a, a variation factor acquisition unit 23b, an adjustment value calculation unit 23c, a determination unit 23d, a measurement schedule determination unit 23e, and a notification unit 23f. Each component of the control unit 23 may be a hardware module, or may be a software module. Processing executed by each component may be regarded as processing executed by the control unit 23.

The intracardiac pressure estimation unit 23a obtains, from the measurement unit 10, cardiac function-related information regarding at least one or more of an electrocardiogram, a pulse wave, and a heart sound via the acquisition unit 21. The intracardiac pressure estimation unit 23a estimates the intracardiac pressure from the measurement value of the obtained cardiac function-related information. The intracardiac pressure to be estimated may include, for example, any one or more of LVEDP, PAP, and PWP. The intracardiac pressure estimation unit 23a may sequentially store, in the storage unit 24, the estimated value of the estimated intracardiac pressure together with time information, such as the time at which the measurement unit 10 has measured the cardiac function-related information.

The intracardiac pressure may be estimated using a trained model generated by machine learning performed with the cardiac function-related information as an input parameter and the intracardiac pressure as an output parameter. The intracardiac pressure estimation unit 23a may estimate the intracardiac pressure by inputting the cardiac function-related information to the trained model.

The variation factor acquisition unit 23b obtains variation factor information that may affect the intracardiac pressure. The variation factor information includes the load information and the time variation information.

The variation factor acquisition unit 23b may obtain the load information from, for example, a load information input unit 22a of the input unit 22. The load information input unit 22a obtains the load information from, for example, the external device 30. The external device 30 can be, for example, an activity meter and/or an acceleration sensor. The variation factor acquisition unit 23b may determine conditions of a posture and/or a motion of the person to be measured from the information regarding the activity amount and/or the acceleration level detected by those devices. Furthermore, the external device 30 may be a blood sugar meter that measures a blood glucose level, for example, and the variation factor acquisition unit 23b may determine whether or not a meal has been taken within the last one to two hours from the change in the blood glucose level. A condition that the person to be measured is in a posture that applies a load on the body, a condition that the person moves the body, a condition that the person has taken a meal, and the like are determined to indicate that the load applied to the body of the person to be measured is high.

The load information input unit 22a is virtually disposed as an element that receives an input of the load information. The input unit 22 may not include dedicated hardware and software for the load information input unit 22a. The load information may be received by the input unit 22, accumulated in the load information storage unit 24a of the storage unit 24, and read by the variation factor acquisition unit 23b as necessary.

The variation factor acquisition unit 23b may use, as the load information, the cardiac function-related information obtained from the measurement unit 10. The variation factor acquisition unit 23b may determine presence or absence of a load on the basis of, for example, an increase in the heart rate and/or an increase in the blood pressure, etc. when compared to a resting state. The increase in the heart rate and/or the increase in the blood pressure of the person to be measured is determined to indicate that the load on the body of the person to be measured is high.

The load information input unit 22a may obtain the load information according to a manual input made by the user. For example, the user may input an elapsed time after a meal, an elapsed time after bathing, or the like using a keyboard, a pointing device, a touch panel, or the like of the input unit 22.

In order to obtain the load information, the variation factor acquisition unit 23b may cause the display unit 25a of the output unit 25 to display guidance for the user on the input of the load information. For example, the variation factor acquisition unit 23b causes the display unit 25a to display guidance that prompts measurement of the load information using the external device 30. Furthermore, for example, the variation factor acquisition unit 23b may cause the display unit 25a to display an input screen for inputting mealtime, bathing time, and the like.

The variation factor acquisition unit 23b may obtain the time variation information from, for example, the time variation information storage unit 24b of the storage unit 24. As described above, the time variation information may be generated by the estimated value of the intracardiac pressure estimated by the intracardiac pressure estimation unit 23a being accumulated in the storage unit 24 together with the time information and being statistically processed. The time variation information storage unit 24b may obtain the time variation information of the intracardiac pressure of a general person from the external server 40 via the input unit 22, and may store it.

The adjustment value calculation unit 23c calculates an adjusted value of the intracardiac pressure obtained by adjusting the intracardiac pressure estimated by the intracardiac pressure estimation unit 23a on the basis of the variation factor information obtained by the variation factor acquisition unit 23b. The adjusted value of the intracardiac pressure is a value obtained by adjusting the estimated value of the intracardiac pressure to the intracardiac pressure at predetermined time measured in a condition where no load is put on the body. The predetermined time may be, for example, predetermined time within a range, for example, from 6:00 a.m. to 8:00 a.m. The intracardiac pressure may be adjusted on the basis of the load applied to the body of the person to be measured and/or the intra-day variation of the intracardiac pressure, etc.

The load applied to the body of the person to be measured may be adjusted on the basis of the estimated value of the intracardiac pressure measured in a resting state with no load. For example, the adjustment value calculation unit 23c formulates an amount of change in the intracardiac pressure based on the presence or absence of a load and the magnitude of the load on the basis of the past data of the person to be measured stored in the storage unit 24, and stores the formulated amount of change in the storage unit 24. At the time of the measurement, the adjustment value calculation unit 23c adjusts the estimated value of the intracardiac pressure using the load applied to the body of the person to be measured at the time of the measurement and the amount of change in the intracardiac pressure with respect to the load stored in the storage unit 24, and calculates the adjusted value indicating the intracardiac pressure when there is no load.

For example, it is assumed that the person to be measured, who normally performs measurement before breakfast, forgets the measurement before meal and performs the measurement after meal. At the time of the measurement of the person to be measured, the user of the intracardiac pressure estimation device 20 manually inputs that the measurement is performed after meal using the input unit 22, for example. Due to the meal taken, a load is applied to the body of the person to be measured so that the intracardiac pressure increases. Thus, the adjustment value calculation unit 23c adjusts the estimated value of the intracardiac pressure obtained as a result of the measurement after meal to the adjusted value of the intracardiac pressure that would have been obtained if the measurement had been performed in a resting state before a meal.

The adjustment based on the intra-day variation of the estimated value of the intracardiac pressure may be carried out on the basis of, for example, an intra-day variation curve obtained from the time variation information of the intracardiac pressure calculated for the person to be measured him/herself. The intra-day variation curve of the intracardiac pressure may be generated in advance by the adjustment value calculation unit 23c on the basis of the time information and the data of the estimated value of the intracardiac pressure of the person to be measured accumulated in the storage unit 24. The estimated value of the intracardiac pressure may be adjusted on the basis of the intra-day variation data of the intracardiac pressure of the general person obtained from the external server 40.

For example, it is assumed that the person to be measured, who normally performs measurement in the morning, forgets the measurement in the morning and performs the measurement at night. In this case, the adjustment value calculation unit 23c specifies an amount of variation in the intracardiac pressure between the nighttime at which the measurement is performed and the morning time at which the measurement is normally performed on the basis of the intra-day variation curve. The adjustment value calculation unit 23c subtracts, adds, multiplies, or divides this amount of variation from the estimated value of the intracardiac pressure, thereby adjusting the estimated value of the intracardiac pressure to a value of the intracardiac pressure that would have been obtained if the measurement had been performed in the morning.

The determination unit 23d determines presence or absence of an abnormality on the basis of a predetermined determination criterion with respect to the adjusted value of the intracardiac pressure calculated by the adjustment value calculation unit 23c. The determination unit 23d may determine the abnormality by setting an absolute threshold or a relative threshold for the adjusted value of the intracardiac pressure by comparison with the adjusted value of the previous intracardiac pressure. For example, the determination unit 23d determines that there is an abnormality when the adjusted value of the intracardiac pressure (e.g., PAP) has become +5 mmHg (i.e., increased by 5 mmHg) or +30% (i.e., increased by 30%) compared to the previous week. Furthermore, for example, the determination unit 23d determines that there is an abnormality when the adjusted value of the intracardiac pressure has become equal to or higher than 25 mmHg.

The determination unit 23d may set the threshold to be used to determine the abnormality on the basis of the patient information obtained through a patient information input unit 22b of the input unit 22. For example, the patient information input unit 22b may receive inputs of the discharge date information, personal medical history, and medical record information of the person to be measured from the medical institution system 50. Those pieces of information may be obtained from the electronic medical record included in the medical institution system 50. Furthermore, the patient information input unit 22b may obtain, for example, a measurement value such as a weight of the patient from a scale, which is one of the external devices 30.

The patient information input unit 22b may obtain the information itself regarding the threshold to be used by the determination unit 23d for the abnormality determination by a manual input by the doctor. Alternatively, the determination unit 23d may automatically set and change the threshold for the abnormality determination on the basis of the patient information input from the patient information input unit 22b. For example, the threshold for the person to be measured who is repeatedly hospitalized in the medical institution is set to be lower (i.e., a lower threshold). Furthermore, for example, the threshold can be set to be lower as the elapsed time after discharge from the medical institution is shorter.

In a similar manner to the load information input unit 22a, the patient information input unit 22b is virtually disposed as an element that receives an input of the patient information. The input unit 22 may not include dedicated hardware and software for the patient information input unit 22b.

In addition to the case where the adjusted value of the intracardiac pressure becomes higher as described above, the cardiac function may be deteriorated when the intra-day variation of the estimated value of the intracardiac pressure becomes smaller. The determination unit 23d may set a threshold for the intra-day variation in the estimated value of the intracardiac pressure, and may determine that an abnormality has occurred when the intra-day variation is equal to or smaller than the threshold. For example, the determination unit 23d may determine that an abnormality has occurred when the intra-day variation in the intracardiac pressure is equal to or lower than 3 mmHg. Furthermore, for example, the determination unit 23d may determine that an abnormality has occurred when the intra-day variation in the intracardiac pressure is equal to or lower than 10% of the maximum value of the estimated value of the intracardiac pressure. In the case of determining the abnormality from the intra-day variation in the estimated value of the intracardiac pressure, the measurement needs to be performed a plurality of times a day using the intracardiac pressure estimation device 20. The user performs the measurement at least twice a day, for example, in the morning and evening.

When it is determined that there is an abnormality, the determination unit 23d may notify the medical institution system 50 of the occurrence of the abnormality by communication via the output unit 25. The doctor of the medical institution may consider how to handle the abnormality of the person to be measured notified to the medical institution system 50.

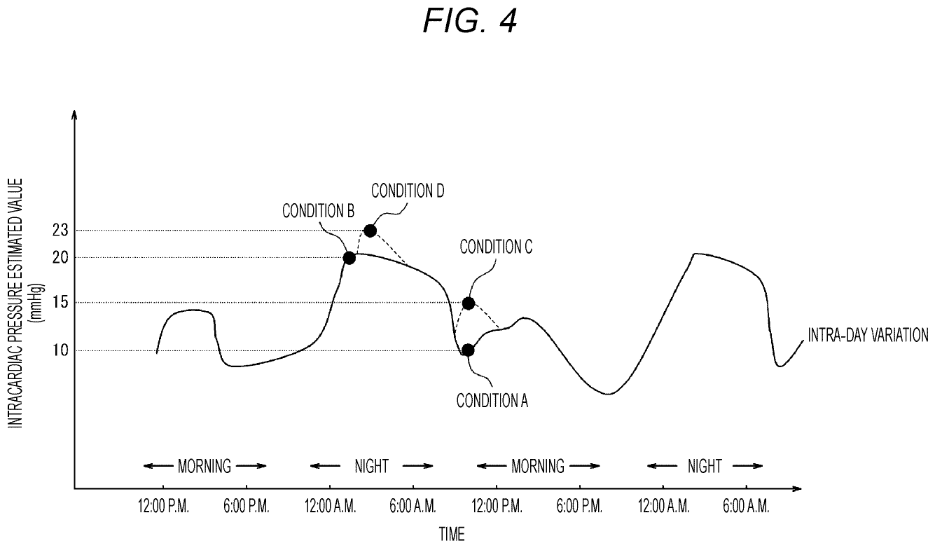

A specific example of the calculation and abnormality determination of the adjusted value of the intracardiac pressure by the control unit 23 will be described with reference to FIG. 4. FIG. 4 is a diagram illustrating variations in the estimated value of the intracardiac pressure according to a load condition and intracardiac pressure measurement time as the time information of the person to be measured in a normal condition. Such a diagram may be calculated on the basis of the past data of the person to be measured accumulated in the past data storage unit 24d of the storage unit 24. A solid line in the drawing indicates an intra-day variation curve when no load is applied to the body of the person to be measured. In addition, a broken line indicates an estimated value of the intracardiac pressure when a load is applied. Condition A indicates that the estimated value of the intracardiac pressure in an unloaded condition in the morning is 10 mmHg. Condition B indicates that the estimated value of the intracardiac pressure in the unloaded condition at night is 20 mmHg. Condition C indicates that the estimated value of the intracardiac pressure in a loaded condition in the morning is 15 mmHg. Condition D indicates that the estimated value of the intracardiac pressure in the loaded condition at night is 23 mmHg. When those are tabulated, the normal intracardiac pressure of the person to be measured is shown in the following table according to the presence or absence of a load and the intracardiac pressure measurement time.

TABLE 1

| | Morning | Night |
|---|---|---|
| Loaded | 15 mmHg | 23 mmHg |
| Unloaded | 10 mmHg | 20 mmHg |

When the intracardiac pressure in the unloaded condition in the morning is set as a reference, the following table regarding the adjustment amount of the estimated value of the intracardiac pressure is obtained. The adjustment amount is used to adjust the estimated value of the intracardiac pressure, and is used to calculate the adjusted value by performing four arithmetic operations, that is, addition, subtraction, multiplication, or division (subtraction in the present embodiment) on the estimated value of the intracardiac pressure.

TABLE 2

| | Morning | Night |
|---|---|---|
| Loaded | +5 mmHg | +13 mmHg |
| Unloaded | Reference | +10 mmHg |

### Example 1

A case is assumed in which the estimated value of the intracardiac pressure in the normal unloaded condition in the morning is 10 mmHg and the estimated value of the intracardiac pressure measured in the unloaded condition at the night of a certain day is 25 mmHg. According to the table above, the intracardiac pressure measured in the unloaded condition at night is +10 mmHg as compared to the reference. Thus, when this estimated value of 25 mmHg of the intracardiac pressure is adjusted to the intracardiac pressure in the unloaded condition in the morning by subtracting 10 mmHg, it is 15 mmHg. This value of 15 mmHg is 5 mmHg larger than the estimated value 10 mmHg of the intracardiac pressure in the unloaded condition in the morning. If a threshold is set to determine an abnormality when an increase of equal to or more than 5 mmHg is recognized in comparison with the normal intracardiac pressure, the control unit 23 determines that an abnormality has occurred.

### Example 2

A case is assumed in which the estimated value of the intracardiac pressure in the normal unloaded condition in the morning is 10 mmHg and the estimated value of the intracardiac pressure measured in the loaded condition after breakfast is 18 mmHg. According to the table above, the intracardiac pressure measured in the loaded condition after breakfast is +5 mmHg as compared to the reference. Thus, when this estimated value of 18 mmHg of the intracardiac pressure is adjusted to the intracardiac pressure in the unloaded condition in the morning by subtracting 5 mmHg, it is 13 mmHg. This value of 13 mmHg is 30% larger than the estimated value 10 mmHg of the intracardiac pressure in the unloaded condition in the morning. If a threshold is set to determine an abnormality when an increase of equal to or more than 30% is recognized in comparison with the normal intracardiac pressure, the control unit 23 determines that an abnormality has occurred.

In the method of calculating the adjusted value of the intracardiac pressure described above, the control unit 23 evaluates the load applied to the body of the person to be measured in two stages of loaded or unloaded condition. However, the control unit 23 may adjust the intracardiac pressure by dividing the magnitude of the load in the case of the loaded condition into two or more stages. For example, the control unit 23 may divide the load in the case of the loaded condition into a first level with a lower load and a second level with a higher load, and may set a different adjustment amount for each of the unloaded condition, the loaded condition of the first level, and the loaded condition of the second level. Furthermore, the control unit 23 may change the adjustment amount depending on a type of the load. For example, the control unit 23 may perform classification such that a load of a light meal is classified into the first level, a load of a heavy meal is classified into the second level, a load of urination is classified into the first level, and a load of defecation is classified into the second level. The control unit 23 may determine the adjustment amount depending on the level of the load. When the load is at the second level, the adjustment amount may be larger than that of the load at the first level.

The measurement schedule determination unit 23e determines a disease risk of the person to be measured on the basis of various conditions in addition to the abnormality determination by the determination unit 23d. The measurement schedule determination unit 23e determines a measurement schedule for measuring the cardiac function-related information and estimating the intracardiac pressure according to the determined risk.

For example, the measurement schedule determination unit 23e classifies the disease risk into a plurality of levels on the basis of the adjusted value of the intracardiac pressure. For example, the measurement schedule determination unit 23e may evaluate the disease risk in three stages according to various criteria to be described below.

### Risk Classification Criterion 1

High risk: The adjusted value of the intracardiac pressure is larger than the threshold (e.g., 30 mmHg)
Moderate risk: The adjusted value of the intracardiac pressure is equal to or smaller than the threshold and shows an upward tendency in the last three days
Low risk: The adjusted value of the intracardiac pressure is equal to or smaller than the threshold and is stable in the last three days

### Risk Classification Criterion 2

High risk: The adjusted value of the intracardiac pressure increases for seven consecutive days
Moderate risk: The adjusted value of the intracardiac pressure increases for three consecutive days
Low risk: The adjusted value of the intracardiac pressure shows no increase

### Risk Classification Criterion 3

High risk: The adjusted value of the intracardiac pressure is larger than a first threshold (e.g., 30 mmHg)
Moderate risk: The adjusted value of the intracardiac pressure is larger than a second threshold (e.g., 20 mmHg)
Low risk: The adjusted value of the intracardiac pressure is equal to or smaller than the second threshold

Moreover, the measurement schedule determination unit 23e may reflect a period of time elapsed after the discharge, the weight, and the like of the person to be measured in the risk determination. For example, those pieces of information may be combined with any one of the risk classifications 1 to 3, and may be used as information for reinforcing the risk level determination.

Risk Classification Criterion 4: Classification based on Elapsed Time Period after Discharge
High risk: Elapse of one month to two months after discharge
Moderate risk: Elapse of two months to six months after discharge
Low risk: Elapse of six months or more after discharge
   Risk Classification Criterion 5: Classification based on Weight Change
High risk: Weight gain of 2 kg or more in one week
Moderate risk: Weight gain of 1 kg or more in one week
Low risk: No weight gain of 1 kg or more in one week

The measurement schedule determination unit 23e may determine the risk level on the basis of the risk classification criteria as described above, and may determine the measurement schedule according to the determined risk level. For example, when it is determined that there is a high level risk according to any one of the risk classification criteria, the measurement schedule determination unit 23e determines the measurement schedule to perform measurement a plurality of times a day including the morning and evening. Furthermore, when there are two or more moderate risks as a result of evaluating the risk according to a plurality of risk classification criteria, the measurement schedule determination unit 23e determines the measurement schedule to perform measurement once a day. Furthermore, when it is determined that there is only a low level risk as a result of evaluating the risk according to the plurality of risk classification criteria, the measurement schedule determination unit 23e sets the measurement schedule to once every two days. Those methods of determining the measurement schedule are merely examples. The measurement schedule determination unit 23e may evaluate the risk by any method, and may determine the measurement schedule by any method. The measurement schedule determination unit 23e may select, as the time for measuring the measurement schedule, a time period in which the person to be measured may stably measure the intracardiac pressure on the basis of the past data accumulated in the past data storage unit 24d of the storage unit 24.

The notification unit 23f notifies the user of the measurement schedule determined by the measurement schedule determination unit 23e using the output unit 25. For example, the notification unit 23f displays the determined measurement schedule on the screen of the display unit 25a. Furthermore, for example, when the measurement time determined in the measurement schedule approaches, the notification unit 23f notifies the user by voice through the speaker 25b. The notification unit 23f may further transmit information to a mobile phone, a wearable device, and/or a television of the user to display the information. When the measurement is not performed at the time determined in the measurement schedule, the notification unit 23f may generate an alarm such as a warning sound to the user.

The notification unit 23f may further transmit the information regarding the measurement schedule to a terminal registered in advance, such as the medical institution system 50 or an information terminal of a family member of the person to be measured, to share the information. As a result, a person related to the person to be measured is enabled to contact the person to be measured or the user at the measurement time to encourage the measurement or the like.

### Flow of Process Executed by Control Unit

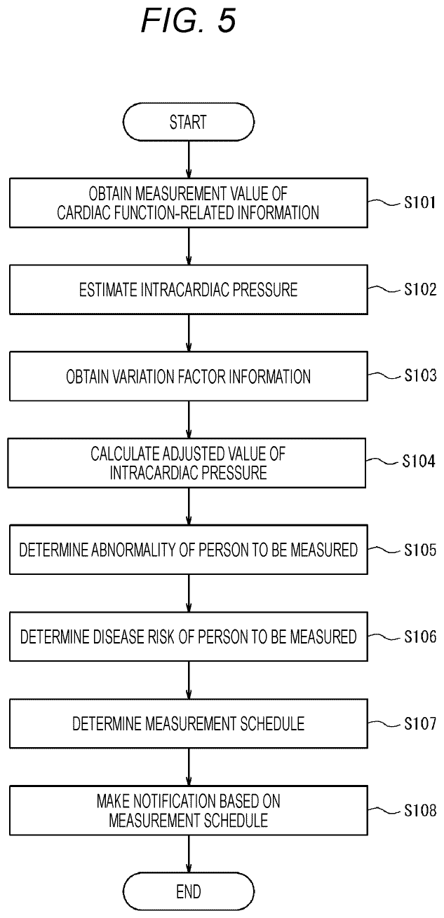

Next, an intracardiac pressure estimation method to be executed by the control unit 23 will be described with reference to a flowchart of FIG. 5. Processing to be executed by each component of the control unit 23 will be described as processing to be executed by the control unit 23. The process of the flowchart of FIG. 5 may be executed by the processor of the control unit 23 in accordance with a program stored in the storage unit 24.

First, the control unit 23 obtains the measurement value of the cardiac function-related information related to the cardiac function measured by the measurement unit 10 via the acquisition unit 21 (step S101).

The control unit 23 estimates the intracardiac pressure on the basis of the measurement value obtained in step S101 (step S102).

The control unit 23 obtains the variation factor information that affects the intracardiac pressure from the input unit 22 and/or the storage unit 24 (step S103). The variation factor information includes the load information that indicates a load applied to the body of the person to be measured. The control unit 23 may cause the output unit 25 to perform display for guiding an input of the load information, and may stand by for the input of the load information to the input unit 22. The variation factor information may further include the time variation information that indicates variations in the intracardiac pressure based on time.

The control unit 23 calculates, on the basis of the variation factor information, the adjusted value of the intracardiac pressure obtained by adjusting the estimated value of the intracardiac pressure to the intracardiac pressure under predetermined conditions (step S104).

The control unit 23 determines the presence or absence of an abnormality in the intracardiac pressure of the person to be measured on the basis of the adjusted value of the intracardiac pressure (step S105). If it is determined that there is an abnormality, the control unit 23 may transmit, to the medical institution system 50 or the like, information indicating the presence of the abnormality, the measurement value by the measurement unit 10, and the estimated value and/or adjusted value of the intracardiac pressure.

The control unit 23 determines the disease risk of the person to be measured on the basis of the adjusted value of the intracardiac pressure and the patient information (step S106). The risk may be evaluated in a plurality of stages, or may be evaluated by scoring.

The control unit 23 determines the measurement schedule for measuring the cardiac function-related information and estimating the intracardiac pressure on the basis of the risk determined in step S106 (step S107). The schedule is set such that the measurement frequency increases as the determined risk is higher.

The control unit 23 makes notification according to the determined schedule (step S108). The control unit 23 displays the measurement schedule on the display unit 25a for the user, and notifies the user when the measurement time in the measurement schedule approaches. Furthermore, when the measurement time has elapsed without performing the measurement, the control unit 23 may issue a warning, for example, by voice or the like using the speaker 25b or the like (step S108).

Note that step S105 and subsequent steps are not necessarily required in the flowchart of FIG. 5. The intracardiac pressure estimation device 20 may perform processing different from steps S105 to S108 using the adjusted value of the intracardiac pressure calculated in step S104. Furthermore, it is not necessary to execute both step S105 and steps S106 to S108, and either one may be executed.

As described above, according to the intracardiac pressure estimation device 20 of the present disclosure, the adjusted value of the intracardiac pressure is calculated on the basis of the variation factor information including the load information indicating the load applied to the body of the person to be measured, whereby the intracardiac pressure may be evaluated in consideration of the influence of the load applied to the person to be measured. Moreover, the intracardiac pressure estimation device 20 of the present disclosure includes the time variation information of the intracardiac pressure in the variation factor information, whereby the intracardiac pressure may be evaluated in consideration of the influence of the time variation of the intracardiac pressure. As a result, for example, even when the measurement is performed at timing different from the normal timing due to forgetting to perform measurement or the like, the intracardiac pressure may be adjusted in consideration of the variations caused by the load applied to the body of the person to be measured and the measurement time, whereby the presence or absence of an abnormality and/or the disease risk may be accurately evaluated.

Furthermore, the intracardiac pressure estimation device 20 may set a threshold for the amount of variation of the intra-day variation in the estimated value of the intracardiac pressure to determine that an abnormality has occurred when the amount of variation is equal to or smaller than the threshold. As a result, it becomes possible to detect a decrease or disappearance of the intra-day variation due to deterioration of the cardiac function, and to grasp the abnormality of the person to be measured.

Furthermore, the intracardiac pressure estimation device 20 of the present disclosure may automatically determine the presence or absence of an abnormality, the disease risk, and the like on the basis of the intracardiac pressure measurement result of the person to be measured, and may contact the medical institution, adjust the number of times of the measurement, and the like. Therefore, the doctor or the like of the medical institution is enabled to easily and appropriately manage the condition of the person to be measured who uses the intracardiac pressure estimation device 20.

Moreover, the intracardiac pressure estimation device 20 may prompt the user to perform measurement at appropriate timing, or may issue a warning when the measurement is delayed. Therefore, the user is enabled to perform measurement in accordance with the notification content of the intracardiac pressure estimation device 20, whereby the user may properly perform the measurement without memorizing the complicated measurement time.

Furthermore, by performing the measurement a plurality of times under different conditions such as different load conditions and/or different timings using the intracardiac pressure estimation device 20, the possibility of detecting the deterioration of the heart failure condition increases.

Although the embodiments according to the present disclosure have been described on the basis of the drawings and examples, it should be noted that those skilled in the art may easily make various modifications or alterations on the basis of the present disclosure. Thus, it should be noted that those modifications or alterations fall within the scope of the present disclosure. For example, the functions and the like included in the individual components of the intracardiac pressure estimation device or the individual steps and the like executed by the control unit of the intracardiac pressure estimation device may be rearranged in a logically consistent manner, and a plurality of the components, steps, and the like may be combined into one or may be divided.

Furthermore, the intracardiac pressure estimation device, the intracardiac pressure estimation system, and the intracardiac pressure estimation method of the present disclosure are not limited to those used at home by the user. The intracardiac pressure estimation device, the intracardiac pressure estimation system, and the intracardiac pressure estimation method of the present disclosure may be used, for example, by a medical worker, such as a doctor, in a medical facility.

The detailed description above describes embodiments of an intracardiac pressure estimation device, an intracardiac pressure estimation system, an intracardiac pressure estimation method, and a program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An intracardiac pressure estimation device comprising:
an acquisition unit configured to obtain cardiac function-related information related to a cardiac function;
an input unit configured to receive an input of load information indicating a load applied to a body of a person to be measured when the cardiac function-related information is measured; and
a control unit configured to estimate an intracardiac pressure that is a pressure applied to an inside of a heart or to a blood vessel near the heart on a basis of the cardiac function-related information, obtain variation factor information that is information indicating a variation factor that may affect the intracardiac pressure and includes the load information, and calculate an adjusted value of the intracardiac pressure according to the variation factor information.

2. The intracardiac pressure estimation device according to claim 1, wherein
the acquisition unit obtains the cardiac function-related information measured by a non-invasive means.

3. The intracardiac pressure estimation device according to claim 1, wherein
the cardiac function-related information includes at least one of an electrocardiogram, a pulse wave, and a heart sound.

4. The intracardiac pressure estimation device according to claim 1, wherein
the intracardiac pressure includes at least one of a left ventricular end-diastolic pressure, a pulmonary arterial pressure, and a pulmonary wedge pressure.

5. The intracardiac pressure estimation device according to claim 1, wherein
the load information includes at least one of biological information of the person to be measured, environmental information, and presence or absence of a daily life activity of the person to be measured and a time elapsed after the activity is performed.

6. The intracardiac pressure estimation device according to claim 1, wherein
the control unit calculates the adjusted value by using an adjustment amount determined on a basis of at least one of a type and magnitude of the load included in the load information for adjustment of an estimated value of the intracardiac pressure estimated on the basis of the cardiac function-related information.

7. The intracardiac pressure estimation device according to claim 1, wherein
the variation factor information includes time variation information indicating a variation in the intracardiac pressure based on time, and the time variation information includes variation information of at least one of an intra-day variation, a daily variation, a weekly variation, a monthly variation, a seasonal variation, and a yearly variation.

8. The intracardiac pressure estimation device according to claim 7, wherein
the time variation information is constructed by the intracardiac pressure of the person to be measured accumulated together with time information.

9. The intracardiac pressure estimation device according to claim 8, wherein
the time variation information includes the variation information of the intra-day variation, and the control unit determines that a disease risk is high when the intra-day variation is smaller than a predetermined threshold.

10. The intracardiac pressure estimation device according to claim 1, wherein
the control unit determines a disease risk on a basis of the adjusted value of the intracardiac pressure.

11. The intracardiac pressure estimation device according to claim 9, wherein
the control unit considers a weight of the person to be measured input to the input unit when the disease risk is determined.

12. The intracardiac pressure estimation device according to claim 9, wherein
the device is used by the person to be measured who has been discharged from a hospital after heart failure treatment, and the control unit considers a period of time elapsed after the discharge of the person to be measured input to the input unit when the disease risk is determined.

13. The intracardiac pressure estimation device according to claim 9, wherein
the control unit determines a schedule for measuring the cardiac function-related information on a basis of the determined disease risk.

14. An intracardiac pressure estimation system comprising:
a measurement device configured to measure cardiac function-related information related to a cardiac function; and
an intracardiac pressure estimation device including an acquisition unit configured to obtain the cardiac function-related information, an input unit configured to receive an input of load information indicating a load applied to a body of a person to be measured when the cardiac function-related information is measured, and a control unit configured to estimate an intracardiac pressure that is a pressure applied to an inside of a heart or to a blood vessel near the heart on a basis of the cardiac function-related information, obtain variation factor information that is information indicating a variation factor that may affect the intracardiac pressure and includes the load information, and calculate an adjusted value of the intracardiac pressure according to the variation factor information.

15. An intracardiac pressure estimation method to be executed by a control unit of a computer, the intracardiac pressure estimation method comprising:
obtaining cardiac function-related information related to a cardiac function;
guiding an input of load information to an input unit, the load information indicating a load applied to a body of a person to be measured when the cardiac function-related information is measured;
receiving the input of the load information from the input unit;
estimating an intracardiac pressure that is a pressure applied to an inside of a heart or to a blood vessel near the heart on a basis of the cardiac function-related information; and
calculating an adjusted value of the intracardiac pressure according to variation factor information that is information indicating a variation factor that may affect the intracardiac pressure and includes the load information.

16. The intracardiac pressure estimation method according to claim 15, further comprising:
obtaining the cardiac function-related information measured by a non-invasive means.

17. The intracardiac pressure estimation method according to claim 15, wherein
the cardiac function-related information includes at least one of an electrocardiogram, a pulse wave, and a heart sound;
the intracardiac pressure includes at least one of a left ventricular end-diastolic pressure, a pulmonary arterial pressure, and a pulmonary wedge pressure; and
the load information includes at least one of biological information of the person to be measured, environmental information, and presence or absence of a daily life activity of the person to be measured and a time elapsed after the activity is performed.

18. The intracardiac pressure estimation method according to claim 15, further comprising:
calculating the adjusted value by using an adjustment amount determined on a basis of at least one of a type and magnitude of the load included in the load information for adjustment of an estimated value of the intracardiac pressure estimated on the basis of the cardiac function-related information.

19. The intracardiac pressure estimation method according to claim 15, wherein the variation factor information includes time variation information indicating a variation in the intracardiac pressure based on time, and the time variation information includes variation information of at least one of an intra-day variation, a daily variation, a weekly variation, a monthly variation, a seasonal variation, and a yearly variation, and the method further comprises:
constructing the time variation information by the intracardiac pressure of the person to be measured accumulated together with time information.

20. A non-transitory computer-readable medium storing program for causing a processor to execute the method according to claim 15.
